(19) United States
(12) Patent Application Publication
(10) Pub. No.: US 2014/0300861 A1
(43) Pub. Date: Oct. 9, 2014

(54) EYE OBSERVATION APPARATUS

(71) Applicant: HOYA CORPORATION, Tokyo (JP)

(72) Inventor: Makio OISHI, Tokyo (JP)

(73) Assignee: HOYA CORPORATION, Tokyo (JP)

(21) Appl. No.: 14/242,069

(22) Filed: Apr. 1, 2014

(30) Foreign Application Priority Data
Apr. 8, 2013 (JP) 2013-080199

(51) Int. Cl. A61B 3/14

(52) U.S. Cl. CPC A61B 3/14
USPC 351/206

(57) ABSTRACT

An eye observation apparatus includes a non-translucent peripheral frame member, a cylindrical light-transmitting member, and an imaging unit. The peripheral frame member includes a tight-fit inner cylindrical surface and a clearance-fit inner cylindrical surface. The cylindrical light-transmitting member includes a tight-fit outer cylindrical surface and a clearance-fit outer cylindrical surface, the tight-fit and clearance-fit outer cylindrical surfaces being coaxial with the tight-fit and clearance-fit inner cylindrical surfaces of the peripheral frame member. A radial clearance between the clearance-fit inner cylindrical surface of the peripheral frame member and the clearance-fit outer cylindrical surface of the cylindrical light-transmitting member is greater than a radial clearance between the tight-fit inner cylindrical surface of the peripheral frame member and the tight-fit outer cylindrical surface of the cylindrical light-transmitting member.

### BACKGROUND OF THE INVENTION

1. Field of the Invention

The present invention relates to an eye observation apparatus (ophthalmic observation apparatus).

2. Description of the Related Art

Eye observation apparatuses are used to examine eye diseases such as glaucoma, retinal detachment and fundal hemorrhage by observing states of retinal blood vessels, retina and optic nerves, etc. In recent years, handheld (portable) eye observation apparatuses for small children and examinees (patients) who have difficulty in placing his or her lower jaw on an examination table (e.g., a chin-rest) have been under development.

The assignee of the present invention has proposed, in Japanese Unexamined Patent Publication No. 2013-202100 (Japanese Patent Application No. 2012-71683), a handheld eye observation apparatus (fundus oculi observation device) in which a non-translucent peripheral frame member, a cylindrical (tubular) light-transmitting member (which guides the illumination light which is incident thereon from the rear forward) and an imaging unit (which includes an imaging portion for imaging retina images) are arranged in that order from outside to inside. Halation and ghosting which may be caused by reflection of illumination light on the surface of the crystalline lens are prevented from occurring by making the cylindrical light-transmitting member out of a translucent member which is brown in tone so that the illumination light becomes brown in tone.

However, according to research by the inventor of the present invention, in the eye observation apparatus disclosed in Japanese Patent Application No. 2012-71683, the outer cylindrical surface of the cylindrical light-transmitting member is engaged with the inner cylindrical surface of the peripheral frame member, over the entire length of the cylindrical light-transmitting member in the axial direction thereof, in a tight-fitting manner (i.e., the cylindrical light-transmitting member is tightly-fitted into the peripheral frame member with the outer cylindrical surface of the cylindrical light-transmitting member brought in contact with the inner cylindrical surface of the peripheral frame member); therefore, illumination light which is guided to the cylindrical light-transmitting member is absorbed by the peripheral frame member, which deteriorates the efficiency of utilization of the illumination light, and accordingly, the eye observation apparatus has a technical problem in which the peripheral frame member generates heat, so that there has been room for improvement in this respect. In this regard, this technical problem holds true similarly for the relationship between the cylindrical light-transmitting member and the imaging unit.

### SUMMARY OF THE INVENTION

The present invention has been achieved based on the above described problems and provides an eye observation apparatus which properly and efficiently guides illumination light in the cylindrical light-transmitting member to ensure appropriate output of the illumination light and which makes it possible to prevent the peripheral frame member, which is positioned outside the cylindrical light-transmitting member, from unexpectedly generating heat.

According to an aspect of the present invention, an eye observation apparatus is provided, including a peripheral frame member which is non-translucent; a cylindrical light-transmitting member which guides illumination light in a forward direction from an incident rear end surface thereof; and an imaging unit which includes an imaging portion for imaging retina images, wherein the peripheral frame member, the cylindrical light-transmitting member and the imaging unit are arranged from outside to inside, in that order. The peripheral frame member includes a tight-fit inner cylindrical surface and a clearance-fit inner cylindrical surface which are formed on an inner peripheral surface of the peripheral frame member in that order from rear to front. The cylindrical light-transmitting member includes a tight-fit outer cylindrical surface and a clearance-fit outer cylindrical surface which are formed on an outer peripheral surface of the cylindrical light-transmitting member, in that order from the rear to the front, the tight-fit outer cylindrical surface and the clearance-fit outer cylindrical surface being coaxial with the tight-fit inner cylindrical surface and the clearance-fit inner cylindrical surface of the peripheral frame member, respectively. A radial clearance between the clearance-fit inner cylindrical surface of the peripheral frame member and the clearance-fit outer cylindrical surface of the cylindrical light-transmitting member is set greater than a radial clearance between the tight-fit inner cylindrical surface of the peripheral frame member and the tight-fit outer cylindrical surface of the cylindrical light-transmitting member so that the illumination light is totally reflected by the clearance-fit outer cylindrical surface of the cylindrical light-transmitting member.

It is desirable for the tight-fit inner cylindrical surface and the clearance-fit inner cylindrical surface of the peripheral frame member to include a large-diameter inner cylindrical surface and a small-diameter inner cylindrical surface, respectively, the small-diameter inner cylindrical surface being smaller in diameter than the large-diameter inner cylindrical surface; and it is desirable for the tight-fit outer cylindrical surface and the clearance-fit outer cylindrical surface of the cylindrical light-transmitting member to include a large-diameter outer cylindrical surface and a small-diameter outer cylindrical surface, respectively, the small-diameter outer cylindrical surface being smaller in diameter than the large-diameter outer cylindrical surface.

It is desirable for the small-diameter inner cylindrical surface of the peripheral frame member to be set greater in axial length than the large-diameter inner cylindrical surface of the peripheral frame member, and for the small-diameter outer cylindrical surface of the cylindrical light-transmitting member to be set greater in axial length than the large-diameter outer cylindrical surface of the cylindrical light-transmitting member.

It is desirable for the cylindrical light-transmitting member to include a large-diameter inner cylindrical surface and a small-diameter inner cylindrical surface which are formed on an inner peripheral surface of the cylindrical light-transmitting member in that order from the rear to the front, the small-diameter inner cylindrical surface of the cylindrical light-transmitting member being smaller in diameter than the large-diameter inner cylindrical surface of the cylindrical light-transmitting member. The imaging unit includes a large-diameter outer cylindrical surface and a small-diameter outer cylindrical surface which are formed on an outer peripheral surface of the imaging unit in that order from the rear to the front, the small-diameter outer cylindrical surface of the imaging unit being smaller in diameter than the large-diameter outer cylindrical surface of the imaging unit. The large-diameter outer cylindrical surface of the imaging unit is coaxially positioned in the large-diameter inner cylindrical surface of the cylindrical light-transmitting member. The small-diameter outer cylindrical surface of the imaging unit is coaxially positioned in the small-diameter inner cylindrical surface of the cylindrical light-transmitting member. A radial clearance between the large-diameter inner cylindrical surface of the cylindrical light-transmitting member and the large-diameter outer cylindrical surface of the imaging unit is set greater than a radial clearance between the small-diameter inner cylindrical surface of the cylindrical light-transmitting member and the small-diameter outer cylindrical surface of the imaging unit so that the illumination light is totally reflected by the large-diameter inner cylindrical surface of the cylindrical light-transmitting member.

It is desirable for the large-diameter inner cylindrical surface of the cylindrical light-transmitting member to be set greater in axial length than the small-diameter inner cylindrical surface of the cylindrical light-transmitting member, and for the large-diameter outer cylindrical surface of the imaging unit to be set greater in axial length than the small-diameter outer cylindrical surface of the imaging unit.

It is desirable for the axial positions of the small-diameter outer cylindrical surface and the large-diameter inner cylindrical surface of the cylindrical light-transmitting member to overlap each other.

It is desirable for the eye observation apparatus to include a diffuser plate which is installed onto a front end surface of the cylindrical light-transmitting member, wherein the diffuser plate diffuses the illumination light which is guided forward by the cylindrical light-transmitting member.

It is desirable for the eye observation apparatus to include a transparent member which is positioned in front of the diffuser plate, wherein the diffuser plate permits the illumination light which is diffused by the diffuser plate to pass therethrough to travel toward the retina.

It is desirable for the eye observation apparatus to include at least one fiber optic light guide which guides the illumination light, wherein a front end of the fiber optic light guide, from which the illumination light emerges, abuts against a rear end surface of the cylindrical light-transmitting member.

In an embodiment, an eye observation apparatus is provided, including a peripheral frame member which is non-translucent; a cylindrical light-transmitting member which guides illumination light in a forward direction from an incident rear end surface thereof; and an imaging unit which includes an imaging portion for imaging retina images, wherein the peripheral frame member, the cylindrical light-transmitting member and the imaging unit being arranged from outside to inside in that order. The cylindrical light-transmitting member includes a clearance-fit inner cylindrical surface and a tight-fit inner cylindrical surface which are formed on an inner peripheral surface of the cylindrical light-transmitting member in that order from rear to front. The imaging unit includes a clearance-fit outer cylindrical surface and a tight-fit outer cylindrical surface which are formed on an outer peripheral surface of the imaging unit in that order from the rear to the front, the clearance-fit outer cylindrical surface and the tight-fit outer cylindrical surface of the imaging unit being coaxial with the clearance-fit inner cylindrical surface and the tight-fit inner cylindrical surface of the cylindrical light-transmitting member, respectively. A radial clearance between the clearance-fit inner cylindrical surface of the cylindrical light-transmitting member and the clearance-fit outer cylindrical surface of the imaging unit is set greater than a radial clearance between the tight-fit inner cylindrical surface of the cylindrical light-transmitting member and the tight-fit outer cylindrical surface of the imaging unit so that the illumination light is totally reflected by the clearance-fit inner cylindrical surface of the cylindrical light-transmitting member.

It is desirable for the clearance-fit inner cylindrical surface and the tight-fit inner cylindrical surface of the cylindrical light-transmitting member to include a large-diameter inner cylindrical surface and a small-diameter inner cylindrical surface, respectively, the small-diameter inner cylindrical surface being smaller in diameter than the large-diameter inner cylindrical surface. The clearance-fit outer cylindrical surface and the tight-fit outer cylindrical surface of the imaging unit comprise a large-diameter outer cylindrical surface and a small-diameter outer cylindrical surface, respectively, the small-diameter outer cylindrical surface being smaller in diameter than the large-diameter outer cylindrical surface.

According to the present invention, an eye observation apparatus is achieved, which properly and efficiently guides illumination light in the cylindrical light-transmitting member to ensure appropriate output of the illumination light and which makes it possible to prevent the peripheral frame member, which is positioned outside the cylindrical light-transmitting member, from unexpectedly generating heat.

The present disclosure relates to subject matter contained in Japanese Patent Application No. 2013-80199 (filed on Apr. 8, 2013) which is expressly incorporated herein by reference in its entirety.

### BRIEF DESCRIPTION OF THE DRAWINGS

The present invention will be described below in detail with reference to the accompanying drawings in which:

FIG. 1 is a cross sectional view of an upper half of the front end of an embodiment of an eye observation apparatus according to the present invention, showing the internal structure thereof;

FIG. 2 is a front elevational view of the eye observation apparatus shown in FIG. 1;

FIG. 3 is a view similar to that of FIG. 1 and illustrates an upper half of the front end of another embodiment of the eye observation apparatus according to the present invention; and

FIG. 4 is a view similar to that of FIG. 1 and illustrates an upper half of the front end of yet another embodiment of the eye observation apparatus according to the present invention.

### DESCRIPTION OF THE PREFERRED EMBODIMENTS

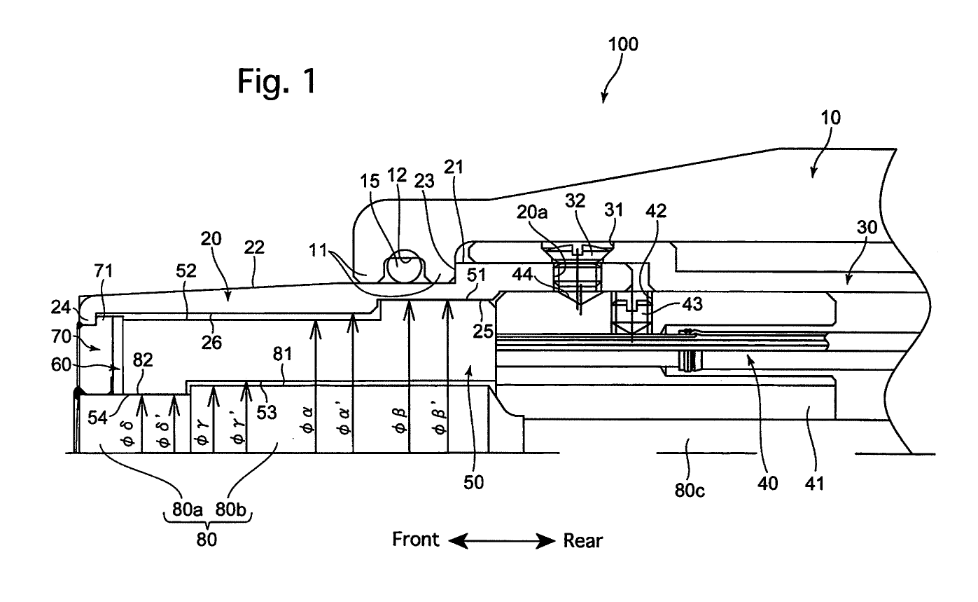

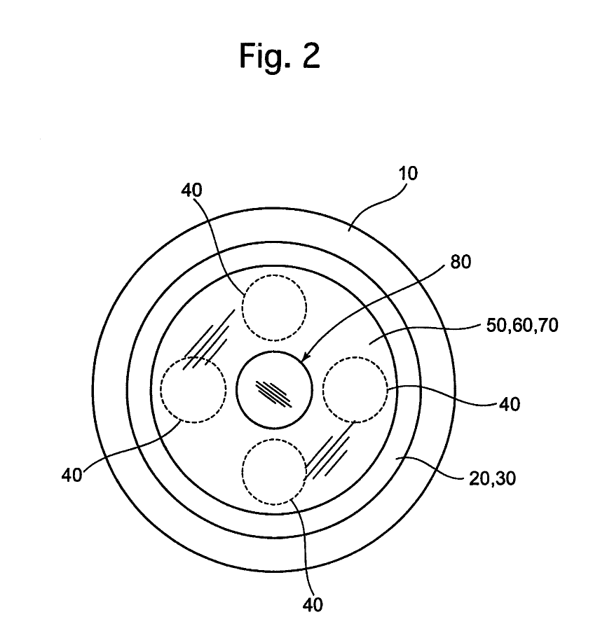

An eye observation apparatus (ophthalmic observation apparatus) 100 according to the present invention will be hereinafter discussed with reference to FIGS. 1 and 2. This embodiment of the eye observation apparatus 100 is of a handheld type (portable type) designed specially for small children or an examinee who is incapable of moving on their own. In the following descriptions, forward and rearward directions are determined with reference to the directions of the double-headed arrows shown in the drawings.

The eye observation apparatus 100 is provided with a holding tube 10 having the shape of a narrow cylinder which is manually held by an operator (e.g., a medial doctor or oculist). The holding tube 10 is configured of a plastic or metal hard tube, or a semi-hard tube which can retain the shape thereof when bent by the operator. The holding tube 10 is formed to have an outer diameter of, e.g., approximately 1 to 2 cm and a length of, e.g., approximately 5 to 15 cm, thus being small in size and easy to handle.

The eye observation apparatus 100 is provided at the front end thereof with a non-translucent peripheral frame member 20 which is made of, e.g., a stainless steel, and the rear end of the peripheral frame member 20 is fitted into the front end of the holding tube 10. An inner flange 11, which projects radially inwards, is formed at the front end of the holding tube 10, and an O-ring retaining groove 12 is formed in the inner flange 11. The O-ring retaining groove 12 is recessed radially outwards and retains an O-ring 15 therein. The peripheral frame member 20 is provided on the outer periphery thereof with a large-diameter cylindrical portion 21 and a small-diameter cylindrical portion 22 in that order from rear to front. The small-diameter cylindrical portion 22 is smaller in diameter than the large-diameter cylindrical portion 21. The peripheral frame member 20 is provided with a stepped portion 23 between the large-diameter cylindrical portion 21 and the small-diameter cylindrical portion 22, and the relative position between holding tube 10 and the peripheral frame member 20 in the axial direction is defined by the stepped portion 23 abutting against the inner flange 11 in the axial direction (horizontal direction with respect to FIG. 1) with the small-diameter cylindrical portion 22 jutting out forwardly from the front end of the holding tube 10. Watertightness between the holding tube 10 and the peripheral frame member 20 (watertightness of the eye observation apparatus 100) is secured by making the O-ring 15, which is retained in the O-ring retaining groove 12, press radially and elastically against the small-diameter cylindrical portion 22.

The eye observation apparatus 100 is provided in the holding tube 10 with an intra-holding-tube frame member 30 which is inserted into the holding tube 10 along the axis direction thereof, and the front end of the intra-holding-tube frame member 30 is connected to the rear end of the peripheral frame member 20. Four radial through-holes 20a are formed in the rear end of the peripheral frame member 20, and corresponding four radial through-holes 31 are formed in the front end of the intra-holding-tube frame member 30. The peripheral frame member 20 and the intra-holding-tube frame member 30 are fixedly connected to each other by screwing four screws 32 into the four radial through-holes 20a through the four radial through-holes 31 with the four radial through-holes 20a and the four through-holes 31 aligned, respectively. The set of four radial through-holes 20a, the set of four radial through-holes 31 and the set of four screws 32 are each arranged at intervals of 90 degrees in the circumferential direction.

The eye observation apparatus 100 is provided inside the peripheral frame member 20 with four fiber optic light guides 40, a cylindrical (tubular) light-transmitting member 50, a diffuser plate 60 and a transparent member 70 which are fitted into the peripheral frame member 20 in that order from rear to front.

The four fiber optic light guides 40 are arranged at intervals of 90 degrees (see FIG. 2) while being supported by a fiber holding frame 41 to guide illumination light forward which is emitted by a light source (a lamp or an LED) which is positioned in the rear end (not shown in the drawings). Four radial through-holes 42 are formed in the fiber holding frame 41, and the four fiber optic light guides 40 and the fiber holding frame 41 are fixed to each other by screwing four screws 43 into the four radial through-holes 42, respectively. In addition, the fiber holding frame 41 is provided, slightly in front of the four radial through-holes 42 on the outer periphery of the fiber holding frame 41, with a circumferential groove 44 into which the four screws 32 are respectively screwed via the four radial through-holes 20a of the peripheral frame member 20 and the four through-holes 31 of the intra-holding-tube frame member 30.

The front end surfaces (exit end surfaces) of the four fiber optic light guides 40 and the fiber holding frame 41 are made to abut against the rear end surface (incident rear end surface) of the cylindrical light-transmitting member 50 to define the relative position of the four fiber optic light guides 40 and the fiber holding frame 41 with respect to the cylindrical light-transmitting member 50 in the axial direction. The cylindrical light-transmitting member 50 guides the illumination light which is incident thereon from the front end of each fiber optic light guide 40 forwardly.

The diffuser plate 60 is made of, e.g., a doughnut-shaped transparent plate provided with a large number of minute pits and projections, on the surface thereof, which are formed to diffuse the illumination light that is incident on the diffuser plate 60 from the front end surface (exit end surface) of the cylindrical light-transmitting member 50.

The transparent member 70 is formed of a cylindrical member which permits the illumination light diffused by the diffuser plate 60 to pass through to travel toward the retina. The transparent member 70 is provided at the rear end thereof with an outer flange 71, the peripheral frame member 20 is provided at the front end thereof with an inner flange 24, and the transparent member 70 is supported and retained by the front end of the peripheral frame member 20 thereat by making the outer flange 71 and the inner flange 24 abut against each other in the axial direction.

The eye observation apparatus 100 is provided therein with an imaging unit 80 which is fitted inside the cylindrical light-transmitting member 50, the diffuser plate 60 and the transparent member 70. The imaging unit 80 is provided with an objective lens unit 80a and a CCD unit (imaging portion) 80b, in that order from front to rear. Images of the retina, as an object image, are passed through the objective lens unit 80a and captured (imaged) by the CCD unit 80b. Retinal images captured by CCD unit 80b are transmitted through a signal cable 80c as image data.

One characteristic feature of the present embodiment of the eye observation apparatus 100 lies in the fit-engagement support structure between the peripheral frame member 20 and the cylindrical light-transmitting member 50. This feature will be discussed hereinafter. The peripheral frame member 20 is provided on the inner peripheral surface thereof with a large-diameter inner cylindrical surface (tight-fit inner cylindrical surface) 25 and a small-diameter inner cylindrical surface (clearance-fit inner cylindrical surface) 26, in that order from rear to front. The small-diameter inner cylindrical surface 26 is smaller in diameter than the large-diameter inner cylindrical surface 25. On the other hand, the cylindrical light-transmitting member 50 is provided on the outer peripheral surface thereof with a large-diameter outer cylindrical surface (tight-fit outer cylindrical surface) 51 and a small-diameter outer cylindrical surface (clearance-fit outer cylindrical surface) 52, in that order from rear to front. The large-diameter outer cylindrical surface 51 is coaxially positioned in the large-diameter inner cylindrical surface 25, and the small-diameter outer cylindrical surface 52 is smaller in diameter than the large-diameter outer cylindrical surface 51 and coaxially positioned in the small-diameter inner cylindrical surface 26. In addition, the radial clearance between the small-diameter inner cylindrical surface 26 of the peripheral frame member 20 and the small-diameter outer cylindrical surface 52 of the cylindrical light-transmitting member 50 is set greater than that between the large-diameter inner cylindrical surface 25 of the peripheral frame member 20 and the large-diameter outer cylindrical surface 51 of the cylindrical light-transmitting member 50. Specifically, the following condition (1) is satisfied:

φα′−φα>φβ′−φβ (1)

wherein φα′ designates the diameter of the small-diameter inner cylindrical surface 26 of the peripheral frame member 20, φα designates the diameter of the small-diameter outer cylindrical surface 52 of the cylindrical light-transmitting member 50, φβ′ designates the diameter of the large-diameter inner cylindrical surface 25 of the peripheral frame member 20, and φβ designates the diameter of the large-diameter outer cylindrical surface 51 of the cylindrical light-transmitting member 50.

The value φβ′−φβ is set to have a positive value as close to zero as possible (i.e., the large-diameter outer cylindrical surface 51 of the cylindrical light-transmitting member 50 is fitted into the large-diameter inner cylindrical surface 25 of the peripheral frame member 20 with a minimum clearance) to ensure the coaxiality between the peripheral frame member 20 and the cylindrical light-transmitting member 50. On the other hand, the value φα′−φα is set, e.g., within the range from 0.025 mm to 2 mm so that the illumination light is totally reflected by the small-diameter outer cylindrical surface 52 of the cylindrical light-transmitting member 50. Therefore, the illumination light which is guided into the cylindrical light-transmitting member 50 is not absorbed by the small-diameter inner cylindrical surface 26 of the peripheral frame member 20, thus being efficiently guided forward in the cylindrical light-transmitting member 50, which ensures an appropriate output of the illumination light and makes it possible to prevent the peripheral frame member 20 from unexpectedly generating heat.

In addition to the above described feature, another characteristic feature of the present embodiment of the eye observation apparatus 100 lies in the fit-engagement support structure between the cylindrical light-transmitting member 50 and the imaging unit 80. This feature will be discussed hereinafter. The cylindrical light-transmitting member 50 is provided on the inner peripheral surface thereof with a large-diameter inner cylindrical surface (clearance-fit inner cylindrical surface) 53 and a small-diameter inner cylindrical surface (tight-fit inner cylindrical surface) 54 in that order from rear to front. The small-diameter inner cylindrical surface 54 is smaller in diameter than the large-diameter inner cylindrical surface 53. On the other hand, the imaging unit 80 is provided on the outer peripheral surface thereof with a large-diameter outer cylindrical surface (clearance-fit outer cylindrical surface) 81 and a small-diameter outer cylindrical surface (tight-fit outer cylindrical surface) 82 in that order from rear to front. The large-diameter outer cylindrical surface 81 is coaxially positioned in the large-diameter inner cylindrical surface 53, and the small-diameter outer cylindrical surface 82 is smaller in diameter than the large-diameter outer cylindrical surface 81 and is coaxially positioned in the small-diameter inner cylindrical surface 54. In addition, the radial clearance between the large-diameter inner cylindrical surface 53 of the cylindrical light-transmitting member 50 and the large-diameter outer cylindrical surface 81 of the imaging unit 80 is set greater than that between the small-diameter inner cylindrical surface 54 of the cylindrical light-transmitting member 50 and the small-diameter outer cylindrical surface 82 of the imaging unit 80. Specifically, the following condition (2) is satisfied:

φγ′−φγ>φδ′−φδ (2)

wherein φγ′ designates the diameter of the large-diameter inner cylindrical surface 53 of the cylindrical light-transmitting member 50, φγ designates the diameter of the large-diameter outer cylindrical surface 81 of the imaging unit 80, φδ designates the diameter of the small-diameter inner cylindrical surface 54 of the cylindrical light-transmitting member 50, and φδ designates the diameter of the small-diameter outer cylindrical surface 82 of the imaging unit 80.

The value φδ′−φδ is set to have a positive value as close to zero as possible (i.e., the small-diameter inner cylindrical surface 54 of the cylindrical light-transmitting member 50 is fitted into the small-diameter outer cylindrical surface 82 of the imaging unit 80 with a minimum clearance) to ensure the coaxiality between the cylindrical light-transmitting member 50 and the imaging unit 80. On the other hand, the value φγ′−φγ is set, e.g., within the range from 0.025 mm to 2 mm so that the illumination light is totally reflected by the large-diameter inner cylindrical surface 53 of the cylindrical light-transmitting member 50. Therefore, the illumination light which is guided into the cylindrical light-transmitting member 50 is not absorbed by the large-diameter outer cylindrical surface 81 of the imaging unit 80, thus being efficiently guided forward in the cylindrical light-transmitting member 50, which ensures appropriate output of the illumination light and makes it possible to prevent the imaging unit 80 from unexpectedly generating heat.

In addition, the present embodiment of the eye observation apparatus 100 is designed so that the small-diameter inner cylindrical surface 26 of the peripheral frame member 20 is set greater in axial length than the large-diameter inner cylindrical surface 25 of the peripheral frame member 20 and so that the small-diameter outer cylindrical surface 52 of the cylindrical light-transmitting member 50 is set greater in axial length than the large-diameter outer cylindrical surface 51 of the cylindrical light-transmitting member 50. Furthermore, the large-diameter inner cylindrical surface 53 of the cylindrical light-transmitting member 50 is set greater in axial length than the small-diameter inner cylindrical surface 54 of the cylindrical light-transmitting member 50, and the large-diameter outer cylindrical surface 81 of the imaging unit 80 is set greater in axial length than the small-diameter outer cylindrical surface 82 of the imaging unit 80. Additionally, the axial positions of the small-diameter outer cylindrical surface 52 and the large-diameter inner cylindrical surface 53 of the cylindrical light-transmitting member 50 overlap each other. This extensively enlarges the range of total reflection of the illumination light by the cylindrical light-transmitting member 50, so that the illumination light is guided forward with better efficiency in the cylindrical light-transmitting member 50, which further ensures an appropriate output of the illumination light and makes it possible to prevent the peripheral frame member 20 and the imaging unit 80, which are positioned inside and outside the cylindrical light-transmitting member 50, respectively, from unexpectedly generating heat.

The eye observation apparatus 100 that has the above described structure is assembled in a manner which will be discussed hereinafter. First, the transparent member 70 and the diffuser plate 60 are inserted into the peripheral frame member 20 and are fitted into the inner peripheral surface thereof in that order with the peripheral frame member 20 facing down. Thereupon, the outer flange 71 of the transparent member 70 abuts against the inner flange 24 of the peripheral frame member 20 in the axial direction, so that the transparent member 70 and the diffuser plate 60 are retained and supported by the front end of the peripheral frame member 20 thereat. Subsequently, the imaging unit 80 is inserted into the cylindrical light-transmitting member 50 to be fitted into and supported by the inner peripheral surface thereof, and a combination of the cylindrical light-transmitting member 50 and the imaging unit 80 is inserted into the peripheral frame member 20 to be fitted into the inner peripheral surface thereof. Subsequently, the front end surfaces of the four fiber optic light guides 40 that are supported by the fiber holding frame 41 are made to abut against the rear end surface of the cylindrical light-transmitting member 50, and the four screws 32 are screwed into the four radial through-holes 20a through the four radial through-holes 31 with the four radial through-holes 20a, which are formed in the rear end of the peripheral frame member 20, and the four through-holes 31, which are formed in the front end of the intra-holding-tube frame member 30, being aligned, respectively, and the four screws 32 are screwed into the circumferential groove 44. Thereupon, an assembly consisting of the peripheral frame member 20, the intra-holding-tube frame member 30, the four fiber optic light guides 40, the cylindrical light-transmitting member 50, the diffuser plate 60, the transparent member 70 and the imaging unit 80 is completed. Lastly, this assembly is inserted into the holding tube 10 to be fitted into the inner peripheral surface thereof. This causes the inner flange 11 of the holding tube 10 and the stepped portion 23 of the peripheral frame member 20 to abut against each other in the axial direction to thereby define the relative position between the holding tube 10 and the peripheral frame member 20, and the O-ring 15, which is retained in the O-ring retaining groove 12, radially comes into contact elastically with the small-diameter cylindrical portion 22 of the peripheral frame member 20, which secures watertightness between the holding tube 10 and the peripheral frame member 20 (watertightness of the eye observation apparatus 100).

When the eye observation apparatus 100 is used, the front end surface of the eye observation apparatus 100 is point to the eye of the examinee, and the aforementioned light source (a lamp or an LED) not shown in the drawings is turned ON to emit illumination light. This illumination light passes through the four fiber optic light guides 40, the cylindrical light-transmitting member 50, the diffuser plate 60 and the transparent member 70, and exits therefrom to reach the retina through the cornea and the crystalline lens of the examinee. The illumination light reflected by the retina is taken in as a retinal image through the objective lens unit 80a and captured by the CCD unit 80b, and the retinal image thus captured is transmitted through the signal cable 80c.

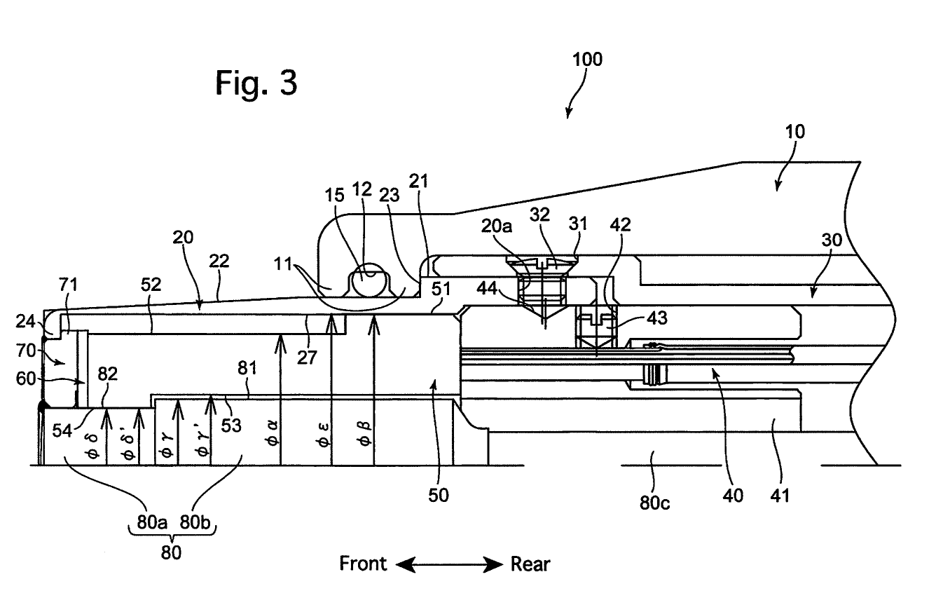

FIG. 3 shows another embodiment of the eye observation apparatus 100 according to the present invention. In this embodiment, the peripheral frame member 20 is provided on the inner peripheral surface thereof with a constant-diameter inner cylindrical surface (tight-fit inner cylindrical surface/clearance-fit inner cylindrical surface) 27 instead of the large-diameter inner cylindrical surface 25 and the small-diameter inner cylindrical surface 26 of the embodiment of the eye observation apparatus 100 shown in FIG. 1. The inside diameter of the constant-diameter inner cylindrical surface 27 is constant throughout from rear to front. The clearance between the constant-diameter inner cylindrical surface 27 of the peripheral frame member 20 and the small-diameter outer cylindrical surface 52 of the cylindrical light-transmitting member 50 is set greater than that between the constant-diameter inner cylindrical surface 27 of the peripheral frame member 20 and the large-diameter outer cylindrical surface 51 of the cylindrical light-transmitting member 50. Specifically, the following condition (3) is satisfied:

φε−φα>φε−φβ (3)

wherein φε designates the diameter of the constant-diameter inner cylindrical surface 27 of the peripheral frame member 20; accordingly, effects similar to those obtained in the above described embodiment shown in FIG. 1 can also be obtained in the embodiment shown in FIG. 3.

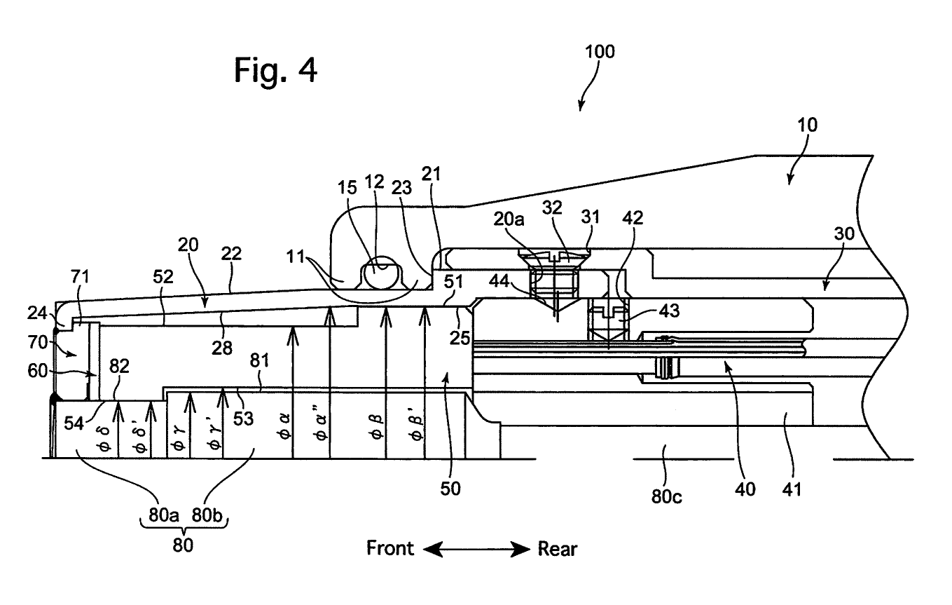

FIG. 4 shows yet another embodiment of the eye observation apparatus 100 according to the present invention. In this embodiment, the peripheral frame member 20 is provided on the inner peripheral surface thereof with a diameter-reducing inner cylindrical surface (clearance-fit inner cylindrical surface) 28 instead of the small-diameter inner cylindrical surface 26 of the embodiment of the eye observation apparatus 100 shown in FIG. 1. The diameter of the diameter-reducing inner cylindrical surface 28 progressively reduces toward the front from the rear. The clearance between the diameter-reducing inner cylindrical surface 28 of the peripheral frame member 20 and the small-diameter outer cylindrical surface 52 of the cylindrical light-transmitting member 50 is set greater than that between the large-diameter inner cylindrical surface 25 of the peripheral frame member 20 and the large-diameter outer cylindrical surface 51 of the cylindrical light-transmitting member 50. Specifically, the following condition (4) is satisfied:

φα″−φα>φβ′−φβ (4)

wherein φα″ designates the diameter of the diameter-reducing inner cylindrical surface 28 of the peripheral frame member 20 at any position in the forward/rearward direction.

Although each of the above described embodiments of the eye observation apparatuses 100 is of a handheld type designed specially for small children and examinees who are incapable of moving on their own, the present invention can also be applied to a stationary type eye observation apparatus in a like manner.

Although the front end surfaces (exit end surfaces) of the four fiber optic light guides 40 are made to abut against the rear end surface (incident end surface) of the cylindrical light-transmitting member 50 in the above described embodiments of the eye observation apparatuses 100, the present invention can also be applied to the case where a clearance is formed between the front end surfaces of the four fiber optic light guides 40 and the rear end surface of the cylindrical light-transmitting member 50.

Obvious changes may be made in the specific embodiments of the present invention described herein, such modifications being within the spirit and scope of the invention claimed. It is indicated that all matter contained herein is illustrative and does not limit the scope of the present invention.

What is claimed is:

1. An eye observation apparatus comprising:
a peripheral frame member which is non-translucent;
a cylindrical light-transmitting member which guides illumination light in a forward direction from an incident rear end surface thereof; and
an imaging unit which includes an imaging portion for imaging retina images, wherein said peripheral frame member, said cylindrical light-transmitting member and said imaging unit are arranged from outside to inside, in that order,
wherein said peripheral frame member includes a tight-fit inner cylindrical surface and a clearance-fit inner cylindrical surface which are formed on an inner peripheral surface of said peripheral frame member in that order from rear to front,
wherein said cylindrical light-transmitting member includes a tight-fit outer cylindrical surface and a clearance-fit outer cylindrical surface which are formed on an outer peripheral surface of said cylindrical light-transmitting member, in that order from said rear to said front, said tight-fit outer cylindrical surface and said clearance-fit outer cylindrical surface being coaxial with said tight-fit inner cylindrical surface and said clearance-fit inner cylindrical surface of said peripheral frame member, respectively, and
wherein a radial clearance between said clearance-fit inner cylindrical surface of said peripheral frame member and said clearance-fit outer cylindrical surface of said cylindrical light-transmitting member is set greater than a radial clearance between said tight-fit inner cylindrical surface of said peripheral frame member and said tight-fit outer cylindrical surface of said cylindrical light-transmitting member so that said illumination light is totally reflected by said clearance-fit outer cylindrical surface of said cylindrical light-transmitting member.

2. The eye observation apparatus according to claim 1, wherein said tight-fit inner cylindrical surface and said clearance-fit inner cylindrical surface of said peripheral frame member comprise a large-diameter inner cylindrical surface and a small-diameter inner cylindrical surface, respectively, said small-diameter inner cylindrical surface being smaller in diameter than said large-diameter inner cylindrical surface, and
wherein said tight-fit outer cylindrical surface and said clearance-fit outer cylindrical surface of said cylindrical light-transmitting member comprise a large-diameter outer cylindrical surface and a small-diameter outer cylindrical surface, respectively, said small-diameter outer cylindrical surface being smaller in diameter than said large-diameter outer cylindrical surface.

3. The eye observation apparatus according to claim 2, wherein said small-diameter inner cylindrical surface of said peripheral frame member is set greater in axial length than said large-diameter inner cylindrical surface of said peripheral frame member, and
wherein said small-diameter outer cylindrical surface of said cylindrical light-transmitting member is set greater in axial length than said large-diameter outer cylindrical surface of said cylindrical light-transmitting member.

4. The eye observation apparatus according to claim 2, wherein said cylindrical light-transmitting member comprises a large-diameter inner cylindrical surface and a small-diameter inner cylindrical surface which are formed on an inner peripheral surface of said cylindrical light-transmitting member in that order from said rear to said front, said small-diameter inner cylindrical surface of said cylindrical light-transmitting member being smaller in diameter than said large-diameter inner cylindrical surface of said cylindrical light-transmitting member,
wherein said imaging unit comprises a large-diameter outer cylindrical surface and a small-diameter outer cylindrical surface which are formed on an outer peripheral surface of said imaging unit in that order from said rear to said front, said small-diameter outer cylindrical surface of said imaging unit being smaller in diameter than said large-diameter outer cylindrical surface of said imaging unit,
wherein said large-diameter outer cylindrical surface of said imaging unit is coaxially positioned in said large-diameter inner cylindrical surface of said cylindrical light-transmitting member,
wherein said small-diameter outer cylindrical surface of said imaging unit is coaxially positioned in said small-diameter inner cylindrical surface of said cylindrical light-transmitting member, and
wherein a radial clearance between said large-diameter inner cylindrical surface of said cylindrical light-transmitting member and said large-diameter outer cylindrical surface of said imaging unit is set greater than a radial clearance between said small-diameter inner cylindrical surface of said cylindrical light-transmitting member and said small-diameter outer cylindrical surface of said imaging unit so that said illumination light is totally reflected by said large-diameter inner cylindrical surface of said cylindrical light-transmitting member.

5. The eye observation apparatus according to claim 4, wherein said large-diameter inner cylindrical surface of said cylindrical light-transmitting member is set greater in axial length than said small-diameter inner cylindrical surface of said cylindrical light-transmitting member, and
wherein said large-diameter outer cylindrical surface of said imaging unit is set greater in axial length than said small-diameter outer cylindrical surface of said imaging unit.

6. The eye observation apparatus according to claim 1, wherein said tight-fit inner cylindrical surface and said clearance-fit inner cylindrical surface of said peripheral frame member comprise a large-diameter inner cylindrical surface and a small-diameter inner cylindrical surface, respectively, said small-diameter inner cylindrical surface being smaller in diameter than said large-diameter inner cylindrical surface,
wherein said tight-fit outer cylindrical surface and said clearance-fit outer cylindrical surface of said cylindrical light-transmitting member comprise a large-diameter outer cylindrical surface and a small-diameter outer cylindrical surface, respectively, said small-diameter outer cylindrical surface being smaller in diameter than said large-diameter outer cylindrical surface,
wherein said small-diameter inner cylindrical surface of said peripheral frame member is set greater in axial length than said large-diameter inner cylindrical surface of said peripheral frame member,
wherein said small-diameter outer cylindrical surface of said cylindrical light-transmitting member is set greater in axial length than said large-diameter outer cylindrical surface of said cylindrical light-transmitting member,
wherein said cylindrical light-transmitting member includes a large-diameter inner cylindrical surface and a small-diameter inner cylindrical surface which are formed on an inner peripheral surface of said cylindrical light-transmitting member in that order from said rear to said front, said small-diameter inner cylindrical surface of said cylindrical light-transmitting member being smaller in diameter than said large-diameter inner cylindrical surface of said cylindrical light-transmitting member,
wherein said imaging unit includes a large-diameter outer cylindrical surface and a small-diameter outer cylindrical surface which are formed on an outer peripheral surface of said imaging unit in that order from said rear to said front, said small-diameter outer cylindrical surface of said imaging unit being smaller in diameter than said large-diameter outer cylindrical surface of said imaging unit,
wherein said large-diameter outer cylindrical surface of said imaging unit is coaxially positioned in said large-diameter inner cylindrical surface of said cylindrical light-transmitting member,
wherein said small-diameter outer cylindrical surface of said imaging unit is coaxially positioned in said small-diameter inner cylindrical surface of said cylindrical light-transmitting member,
wherein a radial clearance between said large-diameter inner cylindrical surface of said cylindrical light-transmitting member and said large-diameter outer cylindrical surface of said imaging unit is set greater than a radial clearance between said small-diameter inner cylindrical surface of said cylindrical light-transmitting member and said small-diameter outer cylindrical surface of said imaging unit so that said illumination light is totally reflected by said large-diameter inner cylindrical surface of said cylindrical light-transmitting member,
wherein said large-diameter inner cylindrical surface of said cylindrical light-transmitting member is set greater in axial length than said small-diameter inner cylindrical surface of said cylindrical light-transmitting member,
wherein said large-diameter outer cylindrical surface of said imaging unit is set greater in axial length than said small-diameter outer cylindrical surface of said imaging unit, and
wherein axial positions of said small-diameter outer cylindrical surface and said large-diameter inner cylindrical surface of said cylindrical light-transmitting member overlap each other.

7. The eye observation apparatus according to claim 1, further comprising a diffuser plate which is installed onto a front end surface of said cylindrical light-transmitting member, wherein said diffuser plate diffuses said illumination light which is guided forward by said cylindrical light-transmitting member.

8. The eye observation apparatus according to claim 7, further comprising a transparent member which is positioned in front of said diffuser plate, wherein said diffuser plate permits said illumination light which is diffused by said diffuser plate to pass therethrough to travel toward said retina.

9. The eye observation apparatus according to claim 1, further comprising at least one fiber optic light guide which guides said illumination light,
wherein a front end of said fiber optic light guide, from which said illumination light emerges, abuts against a rear end surface of said cylindrical light-transmitting member.

10. An eye observation apparatus comprising:
a peripheral frame member which is non-translucent;
a cylindrical light-transmitting member which guides illumination light in a forward direction from an incident rear end surface thereof; and
an imaging unit which includes an imaging portion for imaging retina images, wherein said peripheral frame member, said cylindrical light-transmitting member and said imaging unit being arranged from outside to inside in that order,
wherein said cylindrical light-transmitting member includes a clearance-fit inner cylindrical surface and a tight-fit inner cylindrical surface which are formed on an inner peripheral surface of said cylindrical light-transmitting member in that order from rear to front,
wherein said imaging unit includes a clearance-fit outer cylindrical surface and a tight-fit outer cylindrical surface which are formed on an outer peripheral surface of said imaging unit in that order from said rear to said front, said clearance-fit outer cylindrical surface and said tight-fit outer cylindrical surface of said imaging unit being coaxial with said clearance-fit inner cylindrical surface and said tight-fit inner cylindrical surface of said cylindrical light-transmitting member, respectively, and
wherein a radial clearance between said clearance-fit inner cylindrical surface of said cylindrical light-transmitting member and said clearance-fit outer cylindrical surface of said imaging unit is set greater than a radial clearance between said tight-fit inner cylindrical surface of said cylindrical light-transmitting member and said tight-fit outer cylindrical surface of said imaging unit so that said illumination light is totally reflected by said clearance-fit inner cylindrical surface of said cylindrical light-transmitting member.

11. The eye observation apparatus according to claim 10, wherein said clearance-fit inner cylindrical surface and said tight-fit inner cylindrical surface of said cylindrical light-transmitting member comprise a large-diameter inner cylindrical surface and a small-diameter inner cylindrical surface, respectively, said small-diameter inner cylindrical surface being smaller in diameter than said large-diameter inner cylindrical surface, and
wherein said clearance-fit outer cylindrical surface and said tight-fit outer cylindrical surface of said imaging unit comprise a large-diameter outer cylindrical surface and a small-diameter outer cylindrical surface, respectively, said small-diameter outer cylindrical surface being smaller in diameter than said large-diameter outer cylindrical surface.
